(19) United States
(12) Patent Application Publication
(10) Pub. No.: US 2025/0206864 A1
(43) Pub. Date: Jun. 26, 2025

(54) POLYMER AND COMPOSITION

(71) Applicant: FUJIFILM Corporation, Tokyo (JP)

(72) Inventor: Reiko FUKAGAWA, Kanagawa (JP)

(73) Assignee: FUJIFILM Corporation, Tokyo (JP)

(21) Appl. No.: 19/069,966

(22) Filed: Mar. 4, 2025

Related U.S. Application Data
(63) Continuation of application No. PCT/JP2023/031699, filed on Aug. 31, 2023.

(30) Foreign Application Priority Data
Sep. 29, 2022 (JP) 2022-156110

(51) Int. Cl. C08F 222/24; A61K 8/81; A61Q 17/04; C08F 220/06; C08F 220/38

(52) U.S. Cl. CPC C08F 222/24; A61K 8/81; A61Q 17/04; C08F 220/06; C08F 220/387; C08F 2800/10

(57) ABSTRACT

A polymer and a composition, which have excellent ultraviolet shielding properties and excellent appearance of an aqueous solution obtained when added to water. The polymer has a repeating unit A having a structure represented by Formula (A1) and a repeating unit B having a hydrophilic group.

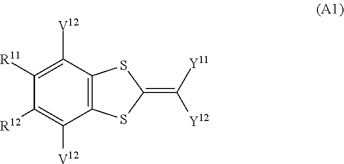

### CROSS-REFERENCE TO RELATED APPLICATIONS

This application is a Continuation of PCT International Application No. PCT/JP2023/031699 filed on Aug. 31, 2023, which was published under PCT Article 21(2) in Japanese, and which claims priority under 35 U.S.C. § 119(a) to Japanese Patent Application No. 2022-156110 filed on Sep. 29, 2022. The above applications are hereby expressly incorporated by reference, in their entirety, into the present application.

### BACKGROUND OF THE INVENTION

### 1. Field of the Invention

The present invention relates to a polymer and a composition.

### 2. Description of the Related Art

An ultraviolet absorber such as an inorganic substance and an organic substance is used for the purpose of shielding ultraviolet rays. The ultraviolet absorber as an organic substance is generally a large number of oil-soluble compounds (for example, polymers), and is often used in combination with an organic solvent. On the other hand, in a case where the ultraviolet absorber is used in combination with an aqueous medium, the ultraviolet absorber may be dissolved in an oily component to form an oil-in-water type emulsified composition.

For example, WO2016/210140A discloses a polymer having a specific structure as an ultraviolet absorber.

### SUMMARY OF THE INVENTION

There is a demand for a polymer which has excellent ultraviolet shielding properties and excellent appearance of an aqueous solution obtained in a case of being added to water, from the viewpoint of environmental load.

The expression that the appearance of the aqueous solution obtained in a case of being added to water is excellent means that the aqueous solution obtained in a case of adding the polymer to water has small turbidity.

As a result of studying a polymer such as that of WO2016/210140A, the present inventors have found that it is difficult to achieve both ultraviolet shielding properties and the appearance of the aqueous solution obtained in a case of being added to water, and there is room for further improvement.

In view of the above circumstances, an object of the present invention is to provide a polymer which has excellent ultraviolet shielding properties and excellent appearance of an aqueous solution obtained in a case of being added to water.

Another object of the present invention is to provide a composition containing the above-described polymer.

As a result of intensive studies on the above-described object, the present inventors have found that the above-described object can be achieved by the following configurations.

[1]

A polymer comprising:
a repeating unit A having a structure represented by Formula (A1) described later; and
a repeating unit B having a hydrophilic group.

[2]

The polymer according to [1],
in which the repeating unit A has a structure represented by Formula (A2) described later.

[3]

The polymer according to [1] or [2],
in which the repeating unit A and the repeating unit B include a repeating unit derived from a monomer having at least one polymerizable group selected from the group consisting of a (meth)acrylic group, a styryl group, a (meth)acrylamide group, and a vinyl ether group.

[4]

The polymer according to [1] or [2],
in which the repeating unit A has a structure represented by Formula (A3) described later.

[5]

The polymer according to any one of [1] to [4],
in which the hydrophilic group included in the repeating unit B is at least one group selected from the group consisting of a carboxylic acid group, a sulfonic acid group, and salts of the groups.

[6]

The polymer according to any one of [1] to [5],
in which the repeating unit B includes a repeating unit derived from at least one monomer selected from the group consisting of (meth)acrylic acid, itaconic acid, β-carboxyethyl (meth)acrylate, 2-(meth)acrylamide-2-methylpropanesulfonic acid, 3-sulfopropyl (meth)acrylate, and salts of the compounds.

[7]

A composition comprising:
the polymer according to any one of [1] to [6]; and water.

According to the present invention, it is possible to provide a polymer which has excellent ultraviolet shielding properties and excellent appearance of an aqueous solution obtained in a case of being added to water.

In addition, according to the present invention, it is possible to provide a composition containing the above-described polymer.

### DESCRIPTION OF THE PREFERRED EMBODIMENTS

In the present specification, numerical ranges represented by “to” include numerical values before and after “to” as lower limit values and upper limit values.

In the present specification, in a case where there are two or more components corresponding to a certain component, “content” of such a component means the total content of the two or more components.

In the present specification, a weight-average molecular weight is a value in terms of polystyrene by gel permeation chromatography (GPC) measurement.

A bonding direction of divalent groups cited in the present specification is not limited unless otherwise specified. For example, in a case where Y in a compound represented by Formula “X—Y—Z” is —COO—, Y may be —CO—O— or —O—CO—. In addition, the above-described compound may be “X—CO—O—Z” or “X—O—CO—Z”.

In the present specification, “ultraviolet rays” mean light having a wavelength range of 10 to 400 nm.

In the present specification, “(meth)acrylic” means at least one of acrylic or methacrylic, “(meth)acrylate” means at least one of acrylate or methacrylate, and “(meth)acryloyl group” means at least one of an acryloyl group or a methacryloyl group.

[Polymer]

The polymer according to the embodiment of the present invention (hereinafter, also referred to as “specific polymer”) has a repeating unit A having a structure represented by Formula (A1) and a repeating unit B having a hydrophilic group.

It is presumed that the specific polymer is excellent in both of ultraviolet shielding properties and appearance of an aqueous solution obtained in a case of being added to water (water solubility of the polymer), because the ultraviolet shielding properties are improved by the repeating unit A included in the specific polymer, and the water solubility of the specific polymer is improved by the repeating unit B.

Hereinafter, the fact that at least one effect of being more excellent in ultraviolet shielding properties or being more excellent in appearance of an aqueous solution obtained in a case where the specific polymer is added to water is obtained is also referred to as “effect of the present invention is more excellent”.

Hereinafter, each repeating unit included in the specific polymer will be described in detail.

### [Repeating Unit A]

The specific polymer has a repeating unit A.

The repeating unit A has a structure represented by Formula (A1).

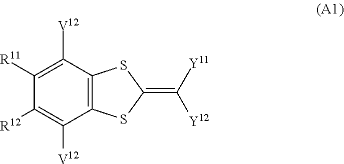

In Formula (A1), one of Y¹¹ or Y¹² represents a cyano group and the other represents a cyano group, an alkylcarbonyl group which may have a substituent, an arylcarbonyl group which may have a substituent, a heterocyclic carbonyl group which may have a substituent, an alkylsulfonyl group which may have a substituent, an arylsulfonyl group which may have a substituent, a carbamoyl group which may have a substituent, a sulfamoyl group which may have a substituent, an alkoxycarbonyl group which may have a substituent, or an aryloxycarbonyl group which may have a substituent,

V¹¹ represents *1-L^{V11}-*2, V¹² represents a hydrogen atom, a monovalent substituent, or *1-L^{V12}-*2 L^{V11} and L^{V12} each independently represent a single bond or a divalent linking group, *1 represents a bonding position to a main chain of the specific polymer, *2 represents a bonding position to a benzene ring specified in Formula (A1), and R¹¹ and R¹² each independently represent a hydrogen atom or a monovalent substituent.

As the alkylcarbonyl group which may have a substituent, represented by Y¹¹ and Y¹², an alkylcarbonyl group having 2 to 8 carbon atoms, which may have a substituent, is preferable; an acetyl group, an ethylcarbonyl group, or a t-butylcarbonyl group is more preferable; and an ethylcarbonyl group or a t-butylcarbonyl group is still more preferable.

As the arylcarbonyl group which may have a substituent, represented by Y¹¹ and Y¹², an arylcarbonyl group having 2 to 14 carbon atoms, which may have a substituent, is preferable; a benzoyl group or a naphthoyl group is more preferable; a benzoyl group is still more preferable.

As the heterocyclic carbonyl group which may have a substituent, represented by Y¹¹ and Y¹², a heterocyclic carbonyl group having 2 to 14 carbon atoms, which may have a substituent, is preferable; a 2-pyridinecarbonyl group or a 2-thiophene carbonyl group is more preferable, and a 2-pyridinecarbonyl group is still more preferable. A heterocyclic ring constituting the above-described heterocyclic carbonyl group may be aromatic or non-aromatic.

As the alkylsulfonyl group which may have a substituent, represented by Y¹¹ and Y¹², an alkylsulfonyl group having 1 to 4 carbon atoms, which may have a substituent, is preferable; and methanesulfonyl is more preferable.

As the arylsulfonyl group which may have a substituent, represented by Y¹¹ and Y¹², an arylsulfonyl group having 6 to 10 carbon atoms, which may have a substituent, is preferable; and benzenesulfonyl is more preferable.

As the carbamoyl group which may have a substituent, represented by Y¹¹ and Y¹², an unsubstituted carbamoyl group or an alkylcarbamoyl group having 1 to 9 carbon atoms, which may have a substituent, is preferable; an unsubstituted carbamoyl group or an alkylcarbamoyl group having 1 to 4 carbon atoms, which may have a substituent, is more preferable; and carbamoyl, N-methylcarbamoyl, N,N-dimethylcarbamoyl, or N-phenylcarbamoyl is still more preferable.

As the sulfamoyl group which may have a substituent, represented by Y¹¹ and Y¹², an alkylsulfamoyl group having 1 to 7 carbon atoms, which may have a substituent, a dialkylsulfamoyl group having 3 to 6 carbon atoms, which may have a substituent, an arylsulfamoyl group having 6 to 11 carbon atoms, which may have a substituent, or a heterocyclic sulfamoyl group having 2 to 10 carbon atoms, which may have a substituent, is preferable; and sulfamoyl, methylsulfamoyl, N,N-dimethylsulfamoyl, phenylsulfamoyl, or 4-pyridinesulfamoyl is more preferable.

As the alkoxycarbonyl group which may have a substituent, represented by Y¹¹ and Y¹², an alkoxycarbonyl group having 2 to 4 carbon atoms, which may have a substituent, is preferable; methoxycarbonyl, ethoxycarbonyl, or (t)-butoxycarbonyl is more preferable; methoxycarbonyl or ethoxycarbonyl is still more preferable; and ethoxycarbonyl is particularly preferable.

As the aryloxycarbonyl group which may have a substituent, represented by Y¹¹ and Y¹², an aryloxycarbonyl group having 6 to 12 carbon atoms, which may have a substituent, is preferable; an aryloxycarbonyl group having 6 to 10 carbon atoms, which may have a substituent, is more preferable; and phenoxycarbonyl, 4-nitrophenoxycarbonyl, 4-acetylaminophenoxycarbonyl, or 4-methanesulfonylphenoxycarbonyl is still more preferable.

Examples of the substituent which can be included in each group represented by Y¹¹ and Y¹² include an alkyl group, an alkoxy group, and an aryl group; and an alkoxy group is preferable.

It is preferable that one of Y¹¹ or Y¹² represents a cyano group and the other represents a cyano group, an alkylcarbonyl group which may have a substituent, an arylcarbonyl group which may have a substituent, a heterocyclic carbonyl group which may have a substituent, a carbamoyl group which may have a substituent, or an alkoxycarbonyl group which may have a substituent; it is more preferable that one of Y¹¹ or Y¹² represents a cyano group and the other represents a cyano group, an alkylcarbonyl group which may have a substituent, an arylcarbonyl group which may have a substituent, a carbamoyl group which may have a substituent, or an alkoxycarbonyl group which may have a substituent; it is still more preferable that one of Y¹¹ or Y¹² represents a cyano group and the other represents a cyano group, an alkylcarbonyl group having 3 to 18 carbon atoms which may have a substituent, an arylcarbonyl group having 7 to 18 carbon atoms which may have a substituent, a carbamoyl group which may have a substituent, or an alkoxycarbonyl group having 3 to 18 carbon atoms which may have a substituent; it is particularly preferable that one of Y¹¹ or Y¹² represents a cyano group and the other represents a cyano group, an ethylcarbonyl group, a t-butylcarbonyl group, a benzoyl group, or an ethoxycarbonyl group; and it is most preferable that Y¹¹ and Y¹² represent a cyano group.

In Formula (A1), V¹¹ represents *1-L^{V11}-*2 L^{V11} represents a single bond or a divalent linking group.

Examples of the divalent linking group represented by L^{V11} include —O—, —S—, —CO—, —COO—, —CONR^{N}—, an alkylene group, an alkenylene group, an arylene group, and a divalent linking group of a combination of these groups. As the divalent linking group of a combination of the groups, —COO-alkylene group-O— or —COO-alkylene group-CO— is preferable; and *1-COO-alkylene group-O-*2 or *1-COO-alkylene group-CO-*2 is more preferable. R^{N} represents a hydrogen atom or a monovalent substituent.

The above-described alkylene group may be linear, branched, or cyclic, and is preferably linear.

The number of carbon atoms in the above-described alkylene group is preferably 1 to 30, more preferably 1 to 10, and still more preferably 1 to 5.

As L^{V11}, *1-X¹—X²—O-*2 or *1-X¹—X²—CO-*2 is also preferable. X¹ and X² have the same meanings as X¹ and X² in Formula (A3), and suitable aspects thereof are also the same.

In Formula (A1), V¹² represents a hydrogen atom, a monovalent substituent, or *1-L^{V12}-*2. L^{V12} represents a single bond or a divalent linking group.

Examples of the monovalent substituent represented by V¹² include a halogen atom, a mercapto group, a cyano group, a carboxy group, a phosphoric acid group, a sulfo group, a hydroxy group, a carbamoyl group, a sulfamoyl group, a nitro group, an alkoxy group, an aryloxy group, an acyl group, an acyloxy group (—OCOR), an acylamino group, a sulfonyl group, a sulfinyl group, a sulfonylamino group, an amino group, an ammonium group, a hydrazino group, a ureido group, an imido group, an alkylthio group, an arylthio group, an alkenylthio group, an alkoxycarbonyl group, an aryloxycarbonyl group, an alkyl group, and an aryl group. The group exemplified as the monovalent substituent represented by V¹² may further have a substituent (for example, a substituent which can be included in Y¹¹ and Y¹²).

As V¹², a cyano group, a nitro group, a hydroxy group, an alkoxy group, an aryloxy group, or an acyloxy group is preferable; an alkoxy group, an aryloxy group, or an acyloxy group is more preferable; an alkoxy group or an acyloxy group is still more preferable; and a methoxy group, an ethoxy group, an i-propyloxy group, a 2-ethylhexyloxy group, a 3,5,5-trimethylhexyloxy group, an acetoxy group, a propionyloxy group, an n-butyloxy group, a t-butyloxy group, a 2-ethylhexyloxy group, a 3,5,5-trimethylhexyloxy group, or a 4-(4-propylcyclohexyl)cyclohexylcarbonyloxy group is particularly preferable.

Examples of the divalent linking group represented by L^{V12} include the divalent linking groups represented by L^{V11}.

As V¹², a monovalent substituent or *1-L^{V12}-*2 is preferable. In addition, in a case where V¹² represents *1-L^{V12}-*2, it is also preferable that L^{V12} represents the same group as L^{V11}.

*1 represents a bonding position to respect to the main chain of the specific polymer. *2 represents a bonding position to the benzene ring specified in Formula (A1).

The benzene ring specified in Formula (A1) in *2 is a benzene ring constituting benzodithiol in Formula (A1), and is a benzene ring to which V¹¹, V¹², R¹¹, and R¹² are directly bonded.

Hereinafter, *1 and *2 will be described in detail with an example of the specific polymer.

For example, in a case where V¹¹ represents *1-COO—(CH₂)₄—O-*2 and V¹² represents a hydrogen atom, an aspect in which the specific polymer has, as the repeating unit A, a repeating unit represented by Formula (PX) is exemplified as an example of the specific polymer. In addition, in a case where V¹¹ represents *1-COO—(CH₂)₄—O-*2 and V¹² represents *1-COO—(CH₂)₄—O-*2, an aspect in which the specific polymer has, as the repeating unit A, a repeating unit represented by Formula (PY) is exemplified as an example of the specific polymer.

In Formula (PX) and Formula (PY), Y¹¹, Y¹², R^{D}, and R¹² each have the same meaning as each notation in Formula (A1).

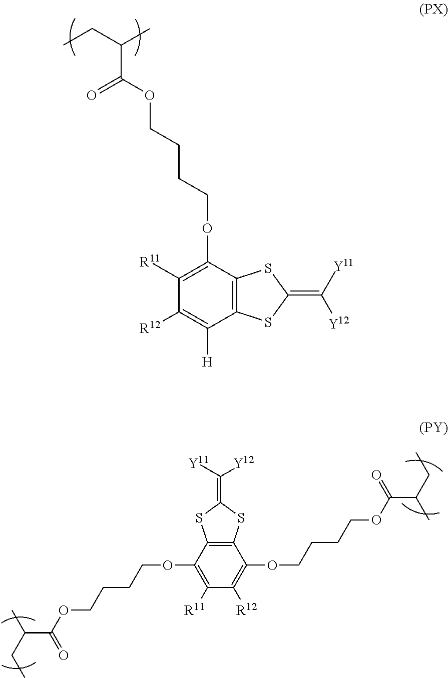

In Formula (A1), R¹¹ and R¹² each independently represent a hydrogen atom or a monovalent substituent.

Examples of the monovalent substituent represented by R¹¹ and R¹² include the monovalent substituents represented by V¹²; and an alkyl group which may have a substituent is preferable, and an unsubstituted alkyl group is more preferable.

The above-described alkyl group may be linear, branched, or cyclic.

The number of carbon atoms in the above-described alkyl group is preferably 1 to 30, more preferably 1 to 10, and still more preferably 1 to 5.

Examples of the above-described alkyl group include a methyl group, an ethyl group, a propyl group, and a butyl group (preferably a t-butyl group).

It is preferable that one of R¹¹ or R¹² represents a hydrogen atom and the other represents a hydrogen atom or an alkyl group which may have a substituent; and it is more preferable that one of R¹¹ or R¹² represents a hydrogen atom and the other represents an alkyl group which may have a substituent.

The repeating unit A preferably has a structure represented by Formula (A2).

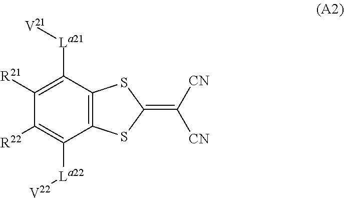

In Formula (A2), V²¹ represents *1-L^{V21}-*2, V²² represents a hydrogen atom, a monovalent substituent, or *1-L^{V22}-*2, L^{V21} and L^{V22} each independently represent a single bond or a divalent linking group, *1 represents a bonding position to a main chain of the specific polymer, *2 represents a bonding position to L^{a21} or L^{a22} specified in Formula (A2),
L^{a21} and L^{a22} each independently represent —O— or —CO—, and
R²¹ and R²² each independently represent a hydrogen atom or a monovalent substituent.

In Formula (A2), V²¹ represents *1-L^{V21}-*2. L^{V21} represents a single bond or a divalent linking group.

Examples of the divalent linking group represented by L^{V21} include the divalent linking groups represented by L^{V11}; and —COO-alkylene group- is preferable and *1-COO-alkylene group-*2 is more preferable.

In Formula (A2), V²² represents a hydrogen atom, a monovalent substituent, or *1-L^{V22}-*2. L^{V22} represents a single bond or a divalent linking group.

Examples of the divalent linking group represented by L^{V22} include the divalent linking groups represented by L^{V11}; and —COO-alkylene group- is preferable and *1-COO-alkylene group-*2 is more preferable.

In a case where V²² represents *1-L^{V22}-*2, it is also preferable that L^{V22} represents the same group as L^{V21}

In Formula (A2), L^{a21} and L^{a22} each independently represent —O— or —CO—.

As L^{a21} and L^{a22}, —O— is preferable. In addition, it is also preferable that L^{a21} and L^{a22} represent the same group.

In Formula (A2), R²¹ and R²² have the same meanings as R¹¹ and R¹², and suitable aspects thereof are also the same.

In Formula (A2), meanings of *1 and *2 can be referred to the meanings of *1 and *2 in Formula (A1).

The repeating unit A also preferably has a structure represented by Formula (A3).

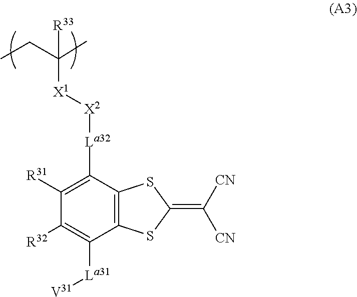

In Formula (A3), V³¹ represents a hydrogen atom, a monovalent substituent, or *1-L^{V3}-*2, L^{V31} represents a single bond or a divalent linking group, *1 represents a bonding position to a main chain of the specific polymer, *2 represents a bonding position to L^{a31} specified in Formula (A3),
L^{a31} and L^{a32} each independently represent —O— or —CO—,
R³¹ and R³² each independently represent a hydrogen atom or a monovalent substituent, R³³ represents a hydrogen atom or a methyl group,
X¹ represents a phenylene group, —COO—, —CONH—, —O—, —CO—, or —CH₂—, and X² represents a single bond or a divalent linking group.

V³¹ has the same meaning as V²², and a suitable aspect thereof is also the same.

L^{a31} and L^{a32} have the same meanings as L^{a21} and L^{a22}, and suitable aspects thereof are also the same.

R³¹ and R³² have the same meanings as R¹¹ and R¹², and suitable aspects thereof are also the same.

In Formula (A3), X¹ represents a phenylene group, —COO—, —CONH—, —O—, or —CO—.

As X¹, a phenylene group, —COO—, or —CONH— is preferable, and —COO— is more preferable.

In Formula (A3), X² represents a single bond or a divalent linking group.

Examples of the divalent linking group represented by X² include the divalent linking groups represented by L^{V22}.

It is also preferable that the repeating unit A includes a repeating unit derived from a monomer having at least one polymerizable group selected from the group consisting of a (meth)acrylic group, a styryl group, a (meth)acrylamide group, and a vinyl ether group.

A content of the repeating unit A is preferably 10% to 99% by mass with respect to the total mass of the specific polymer; and from the viewpoint that the effect of the present invention is more excellent, it is more preferably 30% to 99% by mass, still more preferably 40% to 97% by mass, and particularly preferably 50% to 95% by mass.

### [Repeating Unit B]

The specific polymer has a repeating unit B.

The repeating unit B is a repeating unit having a hydrophilic group.

Examples of the hydrophilic group include a carboxylic acid group and a salt thereof, a sulfonic acid group and a salt thereof, a phosphoric acid group and a salt thereof, and a nonionic hydrophilic group such as a hydroxy group, an amino group, a betaine group, an ethylene glycol group, a polyethylene glycol group, a propylene glycol group, a polypropylene glycol group, and an amide group.

The hydrophilic group is preferably at least one group selected from the group consisting of a carboxylic acid group and a salt thereof, a sulfonic acid group and a salt thereof, and a hydroxy group; and more preferably at least one group selected from the group consisting of a carboxylic acid group and a salt thereof, and a sulfonic acid group and a salt thereof.

The number of hydrophilic groups included in the repeating unit B may be 1, or 2 or more.

The repeating unit B is preferably a repeating unit derived from a monomer having a hydrophilic group and a polymerizable group.

As the polymerizable group, an ethylenically unsaturated group is preferable; a vinyl group, a (meth)acryloyl group, a styryl group, or a maleimide group is more preferable; and a vinyl group or a (meth)acryloyl group is still more preferable.

Examples of a monomer having a carboxylic acid group or a salt thereof and a polymerizable group include acrylic acid, methacrylic acid, crotonic acid, itaconic acid, maleic acid, fumaric acid, citraconic acid, 2-methacryloyloxymethyl succinic acid, β-carboxyethyl acrylate, and salts thereof.

Examples of a monomer having a sulfonic acid group or a salt thereof and a polymerizable group include styrene sulfonic acid, 2-acrylamido-2-methylpropanesulfonic acid, 3-sulfopropyl (meth)acrylate, bis-(3-sulfopropyl)-itaconic acid ester, and salts thereof.

Examples of a monomer having a phosphoric acid group or a salt thereof and a polymerizable group include vinylphosphonic acid, vinylphosphate, bis(methacryloxyethyl)phosphate, diphenyl-2-acryloyloxyethyl phosphate, diphenyl-2-methacryloyloxyethyl phosphate, dibutyl-2-acryloyloxyethyl phosphate, and salts thereof.

Examples of a monomer having a nonionic hydrophilic group and a polymerizable group include ethylenically unsaturated monomers having a (poly)ethyleneoxy group or a polypropyleneoxy group, such as 2-methoxyethyl (meth)acrylate, 2-(2-methoxyethoxy)ethyl (meth)acrylate, ethoxytrethyleneglycol (meth)acrylate, methoxypolyethyleneglycol (molecular weight: 200 to 1000) mono(meth)acrylate, and polyethylene glycol (molecular weight: 200 to 1000) mono(meth)acrylate; and ethylenically unsaturated monomers having a hydroxy group, such as hydroxymethyl (meth)acrylate, 2-hydroxyethyl (meth)acrylate, 2-hydroxypropyl (meth)acrylate, 2,3-dihydroxypropyl (meth)acrylate, 4-hydroxybutyl (meth)acrylate, hydroxypentyl (meth)acrylate, and hydroxyhexyl (meth)acrylate.

The repeating unit B preferably has a repeating unit derived from at least one monomer selected from the group consisting of (meth)acrylic acid, itaconic acid, β-carboxyethyl (meth)acrylate, 2-(meth)acrylamide-2-methylpropane sulfonic acid, 3-sulfopropyl (meth)acrylate, salts thereof, and 2,3-dihydroxypropyl (meth)acrylate; more preferably has a repeating unit derived from at least one monomer selected from the group consisting of (meth)acrylic acid, itaconic acid, β-carboxyethyl (meth)acrylate, 2-(meth)acrylamide-2-methylpropane sulfonic acid, 3-sulfopropyl (meth)acrylate, and salts thereof, and still more preferably has a repeating unit derived from at least one monomer selected from the group consisting of (meth)acrylic acid, β-carboxyethyl (meth)acrylate, 2-(meth)acrylamide-2-methylpropane sulfonic acid, 3-sulfopropyl (meth)acrylate, and salts thereof.

Examples of the salt of the carboxylic acid group, the salt of the sulfonic acid group, and the salt of the phosphoric acid group described above include an alkali metal salt (for example, a lithium salt, a sodium salt, a potassium salt, and the like), an alkaline earth metal salt (for example, a barium salt, a calcium salt, and the like), and an ammonium salt; and an alkali metal salt is preferable.

The repeating unit B is preferably a repeating unit represented by Formula (B).

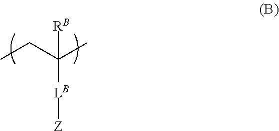

In Formula (B), R^{B} represents a hydrogen atom or a methyl group, L^{B} represents a single bond or a divalent linking group, and Z represents a hydrophilic group.

The hydrophilic group represented by Z is as described above.

Examples of the divalent linking group represented by L^{B} include the divalent linking groups represented by L^{V11}; and —COO—, an alkylene group, —CONR^{N}—, or a divalent linking group of a combination of these groups is preferable. The substituent which can be included in the above-described alkylene group is preferably the hydrophilic group included in the repeating unit B, and more preferably a hydroxy group. R^{N} represents a hydrogen atom or a monovalent substituent.

A content of the repeating unit B is preferably 1% to 90% by mass with respect to the total mass of the specific polymer; and from the viewpoint that the effect of the present invention is more excellent, it is more preferably 1% to 70% by mass, still more preferably 1% to 50% by mass, particularly preferably 5% to 40% by mass, and most preferably 7% to 30% by mass.

The specific polymer may have a repeating unit C other than the repeating unit A and the repeating unit B.

Examples of the repeating unit C include a repeating unit derived from an alkyl (meth)acrylate.

Examples of the specific polymer include the following polymers.

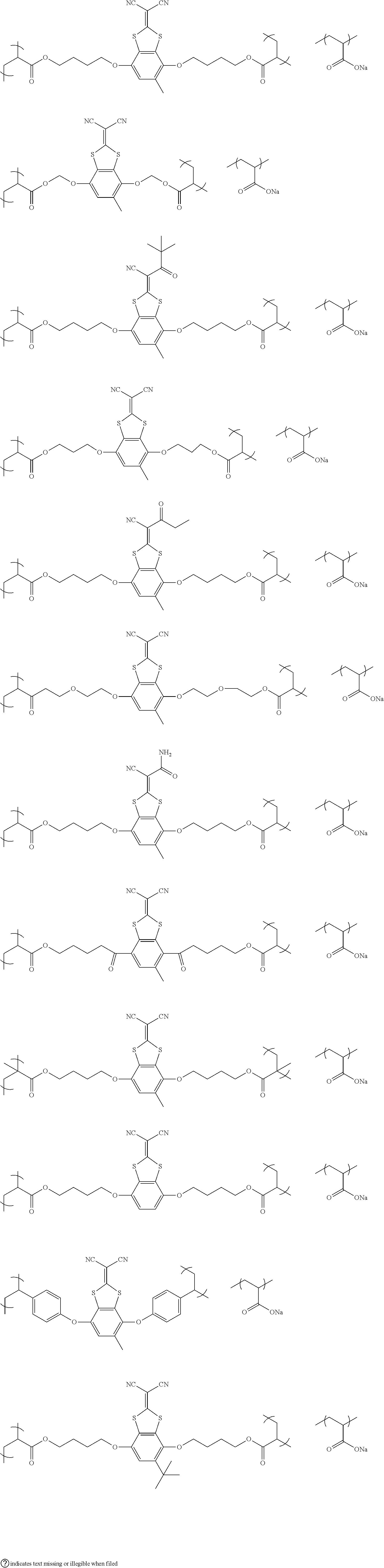

A weight-average molecular weight of the specific polymer is preferably 1,000 to 500,000 and more preferably 1,000 to 100,000; and from the viewpoint that the effect of the present invention is more excellent, it is still more preferably 1,000 to 50,000 and particularly preferably 3,000 to 50,000.

In a case of being within the above-described range, solubility or dispersibility in water is favorable in a case of preparing a composition containing the specific polymer and water, and a viscosity of a cosmetic material described later can be suitably maintained.

The total content of the repeating unit A and the repeating unit B in the specific polymer is preferably 10% to 100% by mass, more preferably 50% to 100% by mass, still more preferably 90% to 100% by mass, and particularly preferably 96% to 100% by mass with respect to the total mass of the specific polymer.

In addition, it is also preferable that the specific polymer has only the repeating unit A and the repeating unit B as repeating units.

A mass ratio of the content of the repeating unit B to the content of the repeating unit A in the specific polymer (content of repeating unit B/content of repeating unit A) is preferably 0.1 to 10, more preferably 0.1 to 3, and still more preferably 0.1 to 1.5.

### [Composition]

The composition contains the specific polymer.

The composition preferably contains the specific polymer and water.

A form of the composition can be appropriately selected according to applications of the composition. Specific examples thereof include an aqueous composition; and a cosmetic material such as an emulsified composition, a gel-like composition, and a solid composition.

The aqueous composition is a composition containing the specific polymer and water, in which a water content is more than 50% by mass with respect to the total mass of the aqueous composition. Among these, it is preferable that the aqueous composition consists of only components selected from the specific polymer, water, and an alkaline compound, contains the specific polymer and water, and has a water content of more than 50% by mass with respect to the total mass of the aqueous composition.

The cosmetic material is a composition other than the aqueous composition, and is a composition containing the specific polymer.

The cosmetic material preferably contains at least one (preferably at least three) selected from the group consisting of an alkali compound, an oil agent, a hydrocarbon, a fatty acid, an alcohol compound, an ester compound, a hydrophilic thickener, a surfactant, and a preservative, which will be described later.

The emulsified composition may be in a form of a lotion or a cream. In addition, an emulsified state may be an oil-in-water droplet (0/W type) or a water-in-oil droplet (W/O type).

In a case where the composition is the emulsified composition, the composition preferably contains the specific polymer, water, and an oil agent.

### [Specific Polymer]

The composition contains the specific polymer.

The specific polymer may be used alone or in combination of two or more kinds thereof.

In a case where the composition is the aqueous composition, a content of the specific polymer is preferably 50.0% to 100.0% by mass, more preferably 90.0% to 100.0% by mass, and still more preferably 95.0% to 100.0% by mass with respect to the total solid content of the aqueous composition. In addition, the content of the specific polymer is preferably 1.0% by mass or more and less than 50.0% by mass, more preferably 5.0% by mass or more and less than 50.0% by mass, and still more preferably 10.0% by mass or more and less than 50.0% by mass with respect to the total mass of the aqueous composition.

In a case where the composition is the cosmetic material, a content of the specific polymer is preferably 0.01% to 45.0% by mass, more preferably 0.1% to 40.0% by mass, and still more preferably 0.5% to 35.0% by mass with respect to the total solid content of the cosmetic material.

In the present specification, the solid content refers to the total mass of components obtained by removing solvents (water and an organic solvent) from all components of the composition.

### [Water]

The composition may contain water.

In a case where the composition contains water, the specific polymer is easily dissolved or dispersed in the composition. In other words, since the specific polymer has the above-described repeating unit B, the appearance of an aqueous solution obtained in a case of being added to water is excellent. Furthermore, in a case where the composition contains water, the specific polymer can be dissolved or dispersed in the composition at a high concentration, and thus the ultraviolet shielding properties can be further improved.

Examples of the water include natural water, purified water, distilled water, deionized water, pure water, and ultrapure water. As the water, from the viewpoint of having few impurities, purified water, distilled water, deionized water, pure water, or ultrapure water is preferable.

In a case where the composition is the aqueous composition, a content of the water is more than 50% by mass, preferably more than 50.0% by mass and 99.0% by mass or less, more preferably more than 50.0% by mass and 95.0% by mass or less, and still more preferably more than 50.0% by mass and 90.0% by mass or less with respect to the total mass of the aqueous composition.

In a case where the composition is the cosmetic material, a content of the water is preferably 10.0% to 80.0% by mass, more preferably 20.0% to 70.0% by mass, and still more preferably 30.0% to 60.0% by mass with respect to the total mass of the cosmetic material.

### [Alkaline Compound]

The composition may contain an alkaline compound.

The alkaline compound is a compound in which an aqueous solution obtained by dissolving the compound in water exhibits alkalinity.

In a case where the composition contains an alkaline compound, the specific polymer is more easily dissolved or dispersed in the composition.

The alkaline compound may be used for forming a salt with the hydrophilic group in the repeating unit B of the specific polymer.

Examples of the alkaline compound include ammonia and salts thereof, such as ammonium hydrogen carbonate; alkanolamine such as monoethanolamine, isopropanolamine, 2-amino-2-methylpropanol, and 2-aminobutanol; and inorganic alkali such as sodium carbonate, potassium carbonate, sodium hydrogen carbonate, and potassium hydrogen carbonate.

### [Oil Agent]

The composition may contain an oil agent.

The oil agent is different from the above-described various components.

Examples of the oil agent include a silicone oil (for example, dimethicone, cyclopentasiloxane, and the like), a hydrocarbon oil (for example, liquid paraffin, vaseline, paraffin wax, squalane, beeswax, carnauba wax, and the like), olive oil, lanolin, a fatty acid, a higher fatty acid, an ester oil (for example, octyldodecyl myristate, ethylhexyl palmitate, isononyl isononanoate, caprylic/capric triglyceride, and the like), ceresine, microcrystalline wax, candelilla wax, diglyceride, triglyceride, jojoba oil, and neopentyl glycol diethylhexanoate.

### [Hydrocarbon]

The composition may contain a hydrocarbon.

The hydrocarbon is different from the above-described various components.

The hydrocarbon is a compound consisting of a carbon atom and a hydrogen atom.

The hydrocarbon may be linear, branched, or cyclic, and is preferably linear or branched.

The hydrocarbon may be saturated or unsaturated. In addition, the hydrocarbon may be an aliphatic hydrocarbon or an aromatic hydrocarbon.

Examples of the hydrocarbon include a mineral oil (for example, light oil, heavy oil, and the like), petrolatum, microcrystalline wax, a paraffinic compound, an isoparaffinic compound, and a hydrogenated isoparaffinic compound; and polydecene, polybutene, hydrogenated polyisobutene, squalane, isohexadecane, or isododecane is preferable.

### [Fatty Acid]

The composition may contain a fatty acid.

The fatty acid is different from the above-described various components.

Examples of the fatty acid include a natural fatty acid synthesized using animal and vegetable oils as raw materials, and linear and branched synthetic fatty acids.

Examples of the fatty acid include lauric acid, myristic acid, palmitic acid, stearic acid, oleic acid, and isostearic acid.

### [Alcohol Compound]

The composition may contain an alcohol compound.

The alcohol compound is different from the above-described various components (for example, the specific polymer).

Examples of the alcohol compound include an aliphatic alcohol, an aromatic alcohol, and saccharides. The aliphatic alcohol is a compound consisting of an aliphatic hydrocarbon of a saturated aliphatic hydrocarbon or an unsaturated aliphatic hydrocarbon, and a hydroxy group (an aliphatic hydrocarbon having a hydroxy group). The aromatic alcohol is a compound consisting of an aromatic hydrocarbon and a hydroxy group (an aromatic hydrocarbon having a hydroxy group). The hydroxy group included in the above-described aromatic alcohol may be an ethereal hydroxy group or a phenolic hydroxy group.

The number of carbon atoms in the alcohol compound is preferably 1 to 40 and more preferably 5 to 30.

Examples of the aliphatic alcohol include monohydric alcohol compounds such as cetyl alcohol, stearyl alcohol, cetearyl alcohol, oleyl alcohol, octyldodecanol, behenyl alcohol, and geranyl alcohol; and polyhydric alcohol compounds such as ethylene glycol, diethylene glycol, triethylene glycol, polyethylene glycol, propylene glycol, glycerin, polyglycerin, 3-methyl-1,3-butanediol, and 1,3-butylene glycol.

Examples of the saccharides include sorbitol, D-sorbitol, glucitol, mannitol, glucose, sucrose, lactose, maltose, maltitol, and trehalose.

### [Ester Compound]

The composition may contain an ester compound.

The ester compound is different from the above-described various components.

The ester compound is a compound obtained by a dehydration reaction between the above-described fatty acid and the above-described alcohol compound (a compound having an ester bond).

Examples of the ester compound include isopropyl myristate, isopropyl palmitate, isopropyl stearate, isopropyl isostearate, isopropyl oleate, n-butyl stearate, n-hexyl laurate, n-decyl oleate, isooctyl stearate, isononyl stearate, isononyl isononanoate, 2-ethylhexyl palmitate, 2-hexyl laurate, 2-hexyldecyl stearate, 2-octyldodecyl palmitate, oleyl oleate, oleyl erucate, elcyl oleate, elcyl erucate, cetyl octanate, cetyl palmitate, cetyl stearate, cetyl oleate, cetyl behenate, cetyl acetate, myristyl myristate, myristyl behenate, myristyl oleate, myristyl stearate, myristyl palmitate, myristyl laurate, myristyl laurate, propylene glycol dicaprylate/dicaprate, stearyl heptanoate, diisostearyl malate, and octyl hydroxy stearate.

### [Hydrophilic Thickener]

The composition may contain a hydrophilic thickener.

Examples of the hydrophilic thickener include a carboxyvinyl polymer (for example, Carbopol, Pemulen, and the like); polyacrylamide (for example, Sepigel 305, SIMULGEL 600, and the like, manufactured by SEPPIC); a polymer and copolymer of 2-acrylamido-2-methylpropanesulfonic acid which may be crosslinked or neutralized (for example, Hostacerin AMPS manufactured by Hoechst AG, and SEIULGEL 800 manufactured by SEPPIC); a copolymer of hydroxyethyl acrylate and 2-acrylamido-2-methylpropanesulfonic acid (for example, SIMULGEL NS, SEPINOV EMT10, and the like, manufactured by SEPPIC); a cellulose derivative (for example, hydroxyethyl cellulose, carboxymethyl cellulose, and the like); polysaccharides (for example, xanthan gum and the like); a synthetic polymer (for example, Intelimer IPA 13-1, Intelimer IPA13-6, and the like, manufactured by Landec); and hectorite and a derivative thereof (for example, BENTONE and the like).

### [Surfactant]

The composition may contain a surfactant.

The surfactant is different from the above-described various components.

Examples of the surfactant include aliphatic alcohol polyglycol ether sulfate, monoglyceride sulfate, mono- and/or di-alkylsulfosuccinate, fatty acid isethionate, fatty acid sarcosinate, fatty acid tauride, fatty acid glutamate, α-olefin sulfonate, ether carboxylic acid, alkyl oligoglucoside, fatty acid glucamide, alkylamide betaine, and a protein fatty acid condensation product.

### [Preservative]

The composition may contain a preservative.

The preservative is different from the above-described various components.

From the viewpoint of preventing contamination of the composition by microorganisms, maintaining the quality, and safety, the composition preferably contains a preservative. The preservative may have a bactericidal action in addition to the preservative action, and may be used for killing or reducing bacteria which proliferate on the skin.

Examples of the bactericidal preservative include benzoic acid, salicylic acid, sorbic acid, paraoxybenzoic acid ester, benzalkonium chloride, benzethonium chloride, halocarbon, 2,4,4-trichloro-2-hydroxyphenol, trichlorocarbanilide, zinc pyrithione, hinokitiol, and phenol.

### [Other Components]

The composition may contain other components in addition to the above-described various components.

Examples of the other components include a polymer other than the specific polymer and an ultraviolet absorber other than the specific polymer. In addition, JP2011-074070A can be referred to, the contents of which are incorporated in the present specification.

In a case where the composition is the cosmetic material, a viscosity of the cosmetic material at 25° C. is preferably 10,000 to 300,000 mPa s, more preferably 12,000 to 200,000 mPa s, and still more preferably 14,000 to 50,000 mPa s.

The viscosity is a value measured under measurement conditions of a rotor No. 4, 12 rpm, and 1 minute using a BL type viscometer.

In a case where the viscosity at 25° C. is 10,000 mPa s or more, stability of the cosmetic material is further improved; and in a case where the viscosity at 25° C. is 300,000 mPa s or less, spreadability during the application of the cosmetic material is mild, and a more favorable use feeling can be obtained.

### [Method for Producing Composition]

Examples of a method for producing the composition include known production methods.

Specific examples thereof include a method for producing a composition, including a step of mixing various components contained in the composition by sequentially adding or collectively adding the various components.

Examples of the mixing step include a method of using a known stirrer.

In the mixing step, a heating treatment may be performed during the mixing.

A temperature in the heating treatment is preferably 50° C. to 95° C. and more preferably 70° C. to 80° C.

### [Applications]

The composition is preferably used in a cosmetic material.

Examples of the cosmetic material include skin care cosmetic materials such as a lotion, a beauty serum, an emulsion, and a cream, a sunscreen preparation, a makeup cosmetic material, a hair care cosmetic material, and a body care cosmetic material.

### EXAMPLES

Hereinafter, the present invention will be described in detail based on Examples. The material, the amount used, the proportion, the process contents, the process procedure, and the like shown in the following examples can be appropriately changed, within a range not departing from the gist of the present invention. Therefore, the scope of the present invention should not be construed as being limited to Examples.

### Example 1

First, a monomer M-1 was synthesized with reference to Synthesis Example 1 of WO2019/131572A.

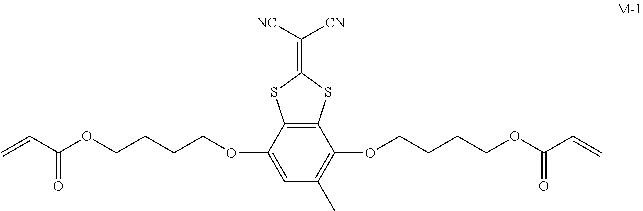 [SMILES: C=CC(=O)OCCCCOc1cc(C)c(OCCCCOC(=O)C=C)c2c1SC(=C(C#N)C#N)S2]

Next, cyclohexanone (12.6 g) and 1-methoxy-2-propanol (12.6 g) were charged into a 300 mL three-neck flask equipped with a stirring blade, a thermometer, a cooling pipe, and a nitrogen introduction pipe, and the mixture was heated to 120° C. under a nitrogen stream. A mixed solution of the monomer M-1 (3.75 g), acrylic acid (1.25 g), a polymerization initiator V-601 (manufactured by FUJIFILM Wako Pure Chemical Corporation) (0.74 g), and cyclohexanone (25.2 g) was added dropwise to the obtained solution over 120 minutes. After aging for 1 hour, a mixed solution of a polymerization initiator V-601 (0.8 g) and cyclohexanone (1.7 g) was further added thereto, and the mixture was aged for 2 hours.

After completion of the reaction, the reaction solution was added dropwise to an excess of hexane, and the precipitated polymer was recovered and air-dried at 60° C. to obtain a polymer (4.46 g). NaHCO₃ (1.40 g) and pure water (40 mL) were added to the obtained polymer, and the mixture was shaken to obtain a composition containing a light yellow transparent polymer A-1. The fact that the target substance was obtained was confirmed by various nuclear magnetic resonance (NMR).

Next, a sunscreen cream (cosmetic material) was prepared.

Various components shown in the table below were stirred at 70° C. to 80° C. for 30 minutes to obtain a uniform solution. The mixture was cooled while being stirred, the stirring was stopped at a moment when the temperature reached 30° C. to 35° C., and the mixture was further allowed to cool to obtain a cosmetic material. The specific polymer was added so that the value in the table was obtained as the solid content.

### Polymer A-1

Emulsifier (NIKKOL NIKKOMULESE 41, manufactured by Nikko Chemicals)
Behenyl alcohol (NIKKOL BEHENYL ALCOHOL 65, manufactured by Nikko Chemicals)
Squalane (NIKKOL SQUALANE, manufactured by Nikko Chemicals)
Tri-2-ethylhexanoic acid glycerol (NIKKOL Trifat 5-308, manufactured by Nikko Chemicals)
Methyl paraben
1,3-Butylene glycol (manufactured by KYOWA HAKKO CHEMICAL CO., LTD.)
Xanthan gum
Purified water

TABLE 1

| Various components | % by mass |
|---|---|
| Polymer A-1 | 15.0 |
| Emulsifier (NIKKOL NIKKOMULESE 41) | 2.5 |
| (emulsifier manufactured by Nikko Chemicals) | |
| Behenyl alcohol (NIKKOL BEHENYL ALCOHOL 65) | 2.5 |
| manufactured by Nikko Chemicals | |
| Squalane (NIKKOL SQUALANE) | 4.0 |
| (manufactured by Nikko Chemicals) | |
| Tri-2-ethylhexanoic acid glycerol (NIKKOL Trifat S-308) | 3.0 |
| (manufactured by Nikko Chemicals) | |
| Methyl paraben | 0.1 |
| 1,3-Butylene glycol | 5.0 |
| (manufactured by KYOWA | |
| HAKKO CHEMICAL CO., LTD.) | |
| Xanthan gum | 15.0 |
| Purified water | 52.9 |

### Examples 2 to 15 and Comparative Examples 1 to 3

In Examples and Comparative Examples other than Example 1, each composition containing a cosmetic material was obtained by the same procedure as in Example 1, except that the polymer shown in the table was synthesized with reference to the synthesis method of the polymer in Example 1.

### [Evaluation]

### [Water Solubility of Specific Polymer (Appearance of Aqueous Solution)]

The polymer and purified water were mixed to prepare an aqueous solution (aqueous composition) having a concentration of solid contents of 15% by mass. The obtained aqueous solution was filled in a 30 mL Nessler tube, shaken, and then visually evaluated according to the following evaluation standard. It was determined that there was no problem in use in a case where the evaluation was 3 or more.
4: no turbidity was observed at all.
3: slight turbidity was observed.
2: turbidity was clearly observed, but the polymer was uniformly dispersed.
1: turbidity was clearly observed, and separation or precipitation occurred.
   [Shielding Rate at Wavelength of 380 nm]

Each cosmetic material was applied onto a PMMA plate (HELIOPLATE H1D6) such that a thin film having a thickness of 10 μm after drying was formed, thereby forming a coating film. A transmission spectrum of the obtained coating film was measured. The transmission spectrum was measured at a wavelength of 380 nm using a UV-VIS spectrophotometer UV-2550 (manufactured by Shimadzu Corporation), and a shielding rate at a wavelength of 380 nm was evaluated as a difference from a case of only the PMMA plate (blank). It was determined that there was no problem in use in a case where the evaluation was 3 or more.
5: shielding rate at a wavelength of 380 nm was 50% or more.
4: shielding rate at a wavelength of 380 nm was 40% or more and less than 50%.
3: shielding rate at a wavelength of 380 nm was 30% or more and less than 40%.
2: shielding rate at a wavelength of 380 nm was 20% or more and less than 30%.
1: shielding rate at a wavelength of 380 nm was less than 20%.

### [Viscosity of Cosmetic Material]

A viscosity of each cosmetic material at 25° C. was measured under measurement conditions of a rotor No. 4, 12 rpm, and 1 minute using a BL-type viscometer (VS-A type, manufactured by Shiaura System Co., Ltd.). It was determined that there was no problem in use in a case where the evaluation was 3 or more.
5: 14,000 to 50,000m Pa s
4: 12,000 mPa s or more and less than 14,000 mPa s, or more than 50,000 mPa s and 200,000 mPa s or less
3: 10,000 mPa s or more and less than 12,000 mPa s, or more than 200,000 mPa s and 300,000 mPa s or less
2: 8,000 mPa s or more and less than 10,000 mPa s, or more than 300,000 mPa s and less than 350,000 mPa s
1: less than 8,000 mPa s or 350,000 mPa s or more

The evaluation results are shown in the following tables.

TABLE 2

| | | | | Evaluation result | | |
|---|---|---|---|---|---|---|
| | Polymer | | | | Shielding | |
| | | | Weight- | | rate at | |
| | | | average | | wave- | |
| | | | mole- | | length | |
| | | | cular | | of 380 | |
| | Type | Structural formula | weight | Appearance | nm | Viscosity |
| Example 1 | A-1 | 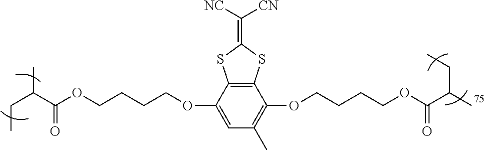 | 7200 | 4 | 5 | 5 |
| | | 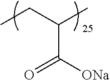 | | | | |
| Example 2 | A-2 | 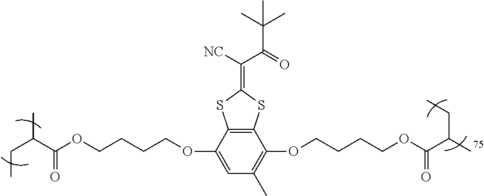 | 9900 | 4 | 5 | 5 |
| | | 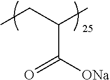 | | | | |
| Example 3 | A-3 | 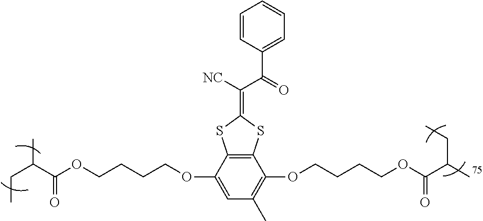 | 14400 | 4 | 5 | 5 |
| | | 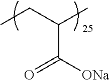 | | | | |
| Example 4 | A-4 | 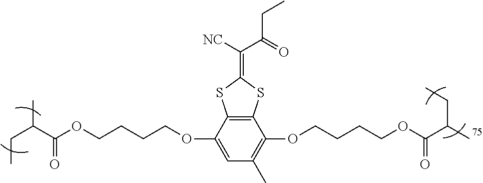 | 11300 | 4 | 5 | 5 |
| | | 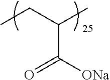 | | | | |
| Example 5 | A-5 | 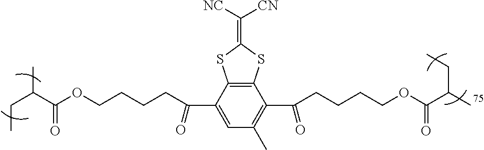 | 13300 | 4 | 5 | 5 |
| | | 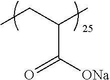 | | | | |
| Example 6 | A-6 | 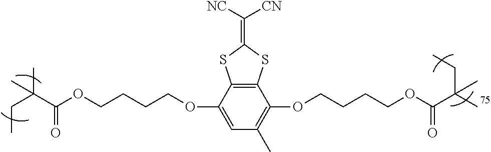 | 6500 | 4 | 5 | 5 |
| | | 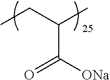 | | | | |
| Example 7 | A-7 | 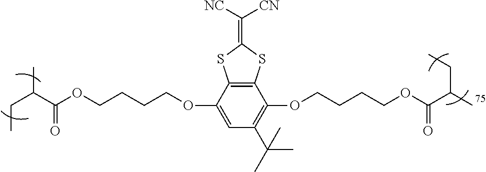 | 25200 | 4 | 5 | 5 |
| | | 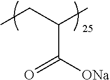 | | | | |
| Example 8 | A-8 | 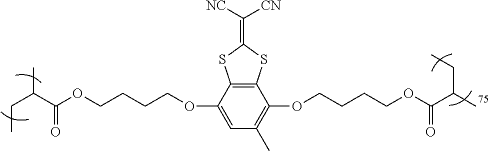 | 8900 | 4 | 5 | 5 |
| | | 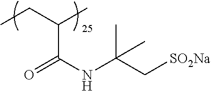 | | | | |
| Example 9 | A-9 | 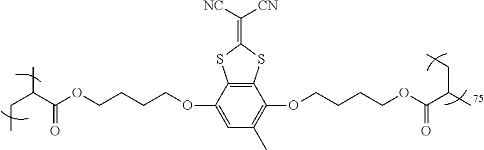 | 7100 | 2 | 5 | 4 |
| | | 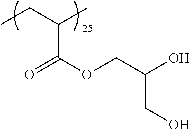 | | | | |

TABLE 3

| | Polymer | | | Evaluation result | | |
|---|---|---|---|---|---|---|
| | | | Weight- | | Shielding | |
| | | | average | | rate at | |
| | | | mole- | | wavelength | |
| | | | cular | Appear- | of 380 | |
| | Type | Structural formula | weight | ance | nm | Viscosity |
| Example 10 | A-10 | 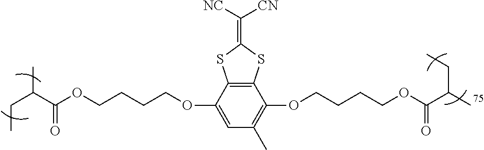 | 14500 | 2 | 5 | 4 |
| | | 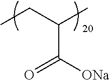 | | | | |
| | | 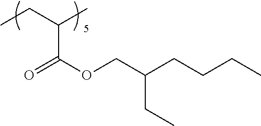 | | | | |
| Example 11 | A-11 | 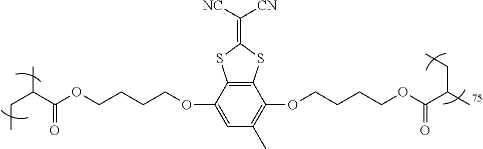 | 1800 | 3 | 5 | 3 |
| | | 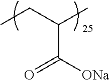 | | | | |
| Example 12 | A-12 | 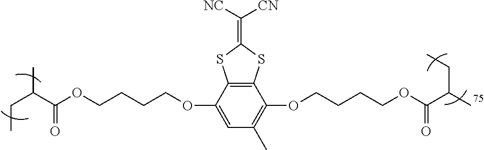 | 65300 | 2 | 5 | 3 |
| | | 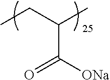 | | | | |
| Example 13 | A-13 | 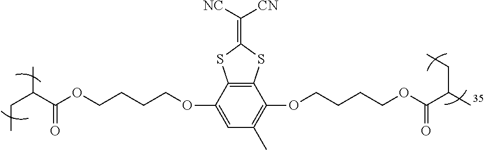 | 11000 | 4 | 4 | 4 |
| | | 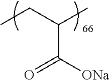 | | | | |
| Example 14 | A-14 | 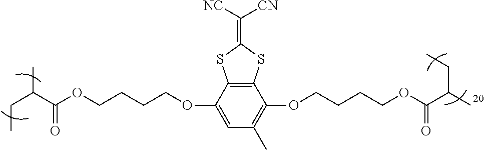 | 13200 | 4 | 3 | 3 |
| | | 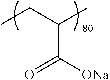 | | | | |
| Example 15 | A-15 | 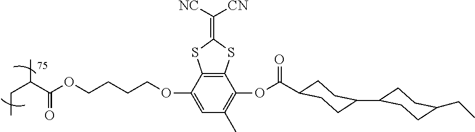 | 16800 | 4 | 5 | 5 |
| | | 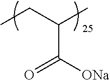 | | | | |
| Comparative Example 1 | a-1 | 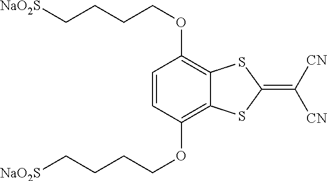 | — | 1 | 5 | 3 |
| Comparative Example 2 | a-2 | 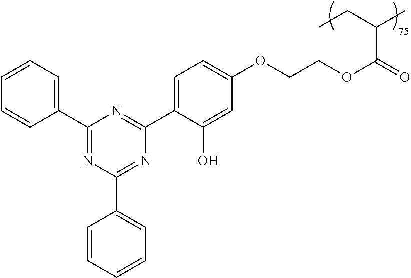 | 6500 | 1 | 2 | 1 |
| | | 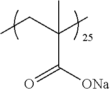 | | | | |
| Comparative Example 3 | a-3 | 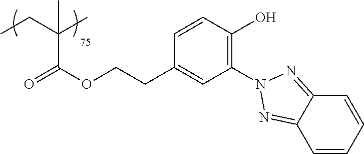 | 9000 | 1 | 1 | 5 |
| | | 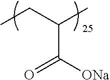 | | | | |

As shown in the tables, it was found that the polymer according to the embodiment of the present invention had excellent ultraviolet shielding properties and excellent appearance of an aqueous solution obtained in a case of being added to water.

It was found that, in a case where the hydrophilic group included in the repeating unit B is at least one group selected from the group consisting of a carboxylic acid group, a sulfonic acid group, and salts thereof, the viscosity and the appearance of the aqueous solution were more excellent (Examples 1 to 9).

It was found that, in a case where the total content of the repeating unit A and the repeating unit B in the specific polymer was 96% to 100% by mass with respect to the total mass of the specific polymer, the viscosity and the appearance of the aqueous solution were more excellent (Examples 1 to 8 and 10).

It was found that, in a case where the weight-average molecular weight of the specific polymer was 1,000 to 50,000, the viscosity and the appearance of the aqueous solution were more excellent (Examples 1 to 8, 11, and 12).

It was found that, in a case where the mass ratio of the content of the repeating unit B to the content of the repeating unit A in the specific polymer (content of repeating unit B/content of repeating unit A) was 0.1 to 3 (preferably 0.1 to 1.5), the ultraviolet shielding properties and the viscosity were more excellent (Examples 1 to 8, 13, and 14).

What is claimed is:

1. A polymer comprising:
a repeating unit A having a structure represented by Formula (A1); and
a repeating unit B having a hydrophilic group, 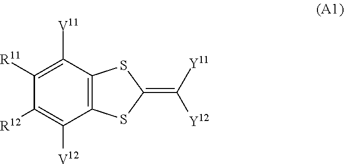
in Formula (A1), one of Y¹¹ or Y¹² represents a cyano group and the other represents a cyano group, an alkylcarbonyl group which may have a substituent, an arylcarbonyl group which may have a substituent, a heterocyclic carbonyl group which may have a substituent, an alkylsulfonyl group which may have a substituent, an arylsulfonyl group which may have a substituent, a carbamoyl group which may have a substituent, a sulfamoyl group which may have a substituent, an alkoxycarbonyl group which may have a substituent, or an aryloxycarbonyl group which may have a substituent,
V¹¹ represents *1-L^{V11}-*2, V¹² represents a hydrogen atom, a monovalent substituent, or *1-L^{V12}-*2 L^{V11} and L^{V12} each independently represent a single bond or a divalent linking group, *1 represents a bonding position to a main chain of the polymer, *2 represents a bonding position to a benzene ring specified in Formula (A1), and
R¹¹ and R¹² each independently represent a hydrogen atom or a monovalent substituent.

2. The polymer according to claim 1,
wherein the repeating unit A has a structure represented by Formula (A2), 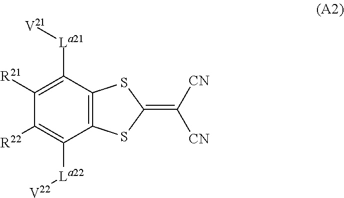
in Formula (A2), V²¹ represents *1-L^{V21}-*2, V²² represents a hydrogen atom, a monovalent substituent, or *1-L^{V22}-*2, L^{V21} and L^{V22} each independently represent a single bond or a divalent linking group, *1 represents a bonding position to a main chain of the polymer, *2 represents a bonding position to L^{a21} or L^{a22} specified in Formula (A2),
L^{a21} and L^{a22} each independently represent —O— or —CO—, and
R²¹ and R²² each independently represent a hydrogen atom or a monovalent substituent.

3. The polymer according to claim 1,
wherein the repeating unit A and the repeating unit B include a repeating unit derived from a monomer having at least one polymerizable group selected from the group consisting of a (meth)acrylic group, a styryl group, a (meth)acrylamide group, and a vinyl ether group.

4. The polymer according to claim 1,
wherein the repeating unit A has a structure represented by Formula (A3), 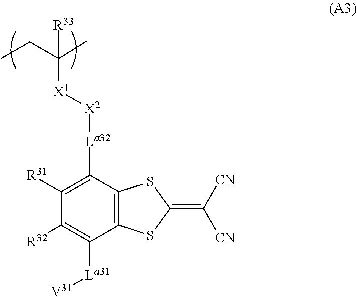
in Formula (A3), V³¹ represents a hydrogen atom, a monovalent substituent, or *1-L^{V31}-*2, L^{V31} represents a single bond or a divalent linking group, *1 represents a bonding position to a main chain of the polymer, *2 represents a bonding position to L³¹ specified in Formula (A3),
L^{a31} and L^{a32} each independently represent —O— or —CO—,
R³¹ and R³² each independently represent a hydrogen atom or a monovalent substituent,
R³³ represents a hydrogen atom or a methyl group,
X¹ represents a phenylene group, —COO—, —CONH—, —O—, —CO—, or —CH₂—, and X² represents a single bond or a divalent linking group.

5. The polymer according to claim 1,
wherein the hydrophilic group included in the repeating unit B is at least one group selected from the group consisting of a carboxylic acid group, a sulfonic acid group, and salts of the groups.

6. The polymer according to claim 1,
wherein the repeating unit B includes a repeating unit derived from at least one monomer selected from the group consisting of (meth)acrylic acid, itaconic acid, β-carboxyethyl (meth)acrylate, 2-(meth)acrylamide-2-methylpropanesulfonic acid, 3-sulfopropyl (meth)acrylate, and salts of the compounds.

7. A composition comprising:
the polymer according to claim 1; and
water.

8. The polymer according to claim 3,
wherein the hydrophilic group included in the repeating unit B is at least one group selected from the group consisting of a carboxylic acid group, a sulfonic acid group, and salts of the groups.

9. The polymer according to claim 3,
wherein the repeating unit B includes a repeating unit derived from at least one monomer selected from the group consisting of (meth)acrylic acid, itaconic acid, β-carboxyethyl (meth)acrylate, 2-(meth)acrylamide-2-methylpropanesulfonic acid, 3-sulfopropyl (meth)acrylate, and salts of the compounds.

10. A composition comprising:
the polymer according to claim 3; and
water.

11. The polymer according to claim 5,
wherein the repeating unit B includes a repeating unit derived from at least one monomer selected from the group consisting of (meth)acrylic acid, itaconic acid, β-carboxyethyl (meth)acrylate, 2-(meth)acrylamide-2-methylpropanesulfonic acid, 3-sulfopropyl (meth)acrylate, and salts of the compounds.
